(19) United States
(12) Patent Application Publication
(10) Pub. No.: US 2008/0191050 A1
(43) Pub. Date: Aug. 14, 2008

(54) Volatile Liquid Dispensing Device

(75) Inventors: Philippe Blondeau, Paris (FR); Alice Bresson Boil, Herblay (FR)

Correspondence Address: NORRIS, MCLAUGHLIN & MARCUS, 875 THIRD AVE, 18TH FLOOR, NEW YORK, NY, 10022, US

(73) Assignee: GIVAUDAN SA, Vernier (CH)

(21) Appl. No.: 11/915,681

(22) PCT Filed: May 31, 2006
(86) PCT No.: PCT/CH2006/000287

§ 371 Date: Nov. 27, 2007

(30) Foreign Application Priority Data
Jun. 2, 2005 (EP) 05291191.4

(51) Int. Cl. A61L 9/00; A61L 9/015; B67D 5/08

(52) U.S. Cl. 239/6; 239/34; 239/71

(57) ABSTRACT

An apparatus (1) adapted to disseminate a volatile liquid (4), such as a fragrance, into an ambient atmosphere for a desired period of time, the apparatus comprising a reservoir (2) having a single opening (3) to the atmosphere, this opening being closed by a permeable membrane (5) not in contact with the liquid in the reservoir when the apparatus is in operation, the membrane having a thickness of from 0.1-5 mm and being capable of absorbing a quantity of liquid that will evaporate over the desired period when the membrane is brought into direct contact with the liquid. The membrane may also act as a end-of-life indicator by changing colour as the liquid is disseminated. The apparatus is an easy-to-use, spill-proof device, with the ability to control the amount of liquid in an atmosphere and to prevent habituation to the liquid.

This invention relates to an apparatus for dispensing volatile substances, and more particularly to a membrane-based dispensing device for the delivery of volatile substances from a liquid to an ambient environment by evaporation.

Membrane-based dispensing devices for the dispensing into an ambient environment of volatile liquids such as fragrances, bactericides, fungicides and disinfectants are well known in the art. One very common type of such dispensing devices consists essentially of a reservoir containing the volatile liquid and a membrane covering the container and contacting the volatile liquid. Such dispensing devices employ diffusion phenomenon to provide the motive dispensing force. The liquid phase evaporates through the membrane to the ambient environment. Such a device may additionally comprise auxiliary dispensing means, such as heating elements and/or fans.

While such devices are undoubtedly successful and have been commercially successful, they have certain practical drawbacks. One is a phenomenon called “habituation”, that is, with continuous emission, people simply get used to the odour and cease to notice it. Much of the fragrance is therefore wasted to a certain extent. This can be overcome, but means of doing this have hitherto been both relatively complex and relatively expensive. For example, it is possible to provide programmable devices with automatically opening orifices and the like. Such expense and complexity is often not justified.

It has now been found that it is possible to provide a simple device that overcomes this problem and allows the release of volatile liquid for a desired period, but without requiring complex means for achieving this. The invention therefore provides an apparatus adapted to disseminate a volatile liquid into an ambient atmosphere for a desired period of time, the apparatus comprising a reservoir having a single opening to the atmosphere, this opening being closed by a permeable membrane not in contact with the liquid in the reservoir when the apparatus is in operation, the membrane having a thickness of from 0.1-5 mm and being capable of absorbing a quantity of liquid that will evaporate over the desired period when the membrane is brought into direct contact with the liquid.

The invention additionally provides a method of disseminating a volatile liquid into an ambient atmosphere for a desired time, comprising the application of the liquid to one side of a porous membrane permanently not exposed to the atmosphere, the membrane having a thickness of from 0.1-5 mm and being capable of absorbing sufficient liquid for evaporation from a side of the membrane exposed to the atmosphere over the desired time period.

Provided that the reservoir has the desired single opening, it may have any suitable shape and be made from any suitable material. Naturally it must be resistant to the volatile liquid contained therein, i.e., not be chemically degraded, softened or swollen by it. Glass, ceramics, metals and selected plastics may be used, any such selection being within the skill of the art.

The membrane closing the opening must not have contact with the liquid in the reservoir when the apparatus is in operation, that is, the two must be physically separate. It therefore follows that the opening must be located in a place on the reservoir where such contact does not occur. Generally, it means that the opening is located at or near the top of the reservoir, when the device is sitting on a horizontal surface, but it can also be in a side of the vessel, provided that the opening has no direct contact with the liquid in the reservoir when the apparatus is operating.

The membrane may be any membrane that meets the following requirements:
it must permit liquid to pass through from that side of the membrane exposed to the interior of the reservoir to that exposed to the atmosphere;
it must be of such a constitution that, when brought into contact with the liquid, it can absorb sufficient liquid for evaporation into the atmosphere for the desired time.

The second of these conditions dictates that the membrane must be of a reasonable thickness—very thin membranes of the type currently used in the art will allow the passage of the liquid, but they will not be able to retain sufficient liquid for evaporation for a significant time. The thickness of the membrane will depend on the natures of the liquid and the membrane, but typical thicknesses range from 0.1-5 mm (compared to the typical 50 μM of the art). It is possible to use thicker membranes, but these become more impractical and more expensive to manufacture. In particular embodiments, the thickness lies between 0.5 and 5 mm, 0.2 and 3 mm, 0.6 and 2 mm and 0.6 and 11.0 mm. The provision of a membrane that holds the desired quantity of a given liquid is well within the skill of the art.

Any material that fulfils these requirements may be used in this invention. One particular membrane material is a polymeric material, in particular a microporous, filled polymeric material. In one particular embodiment, the material is polyolefin. Such materials are commercially available as battery separators. A typical such material consists essentially of a homogeneous mixture of 8 to 100 vol. % polyolefin having a molecular weight (weight-average) of at least 300,000, a standard load melt index of substantially 0 and a reduced viscosity of not less than 4.0, 1 to 92 vol. % filler and 1 to 40 vol. % plasticizer.

Typical suitable materials are described in detail in U.S. Pat. No. 3,351,495. The polyolefin described therein is an ultra-high molecular weight polyolefin, in particular, ultra-high molecular weight polyethylene. It has an average weight-average molecular weight of at least 300,000, preferably at least 1,000,000, and in particular about 4 to 7×10⁶. The standard load melt index of the polyolefin is substantially 0, i.e. it is less than 0.1, and more particularly less than 0.01. The reduced viscosity of the polyolefin is not less than 4.0, and in other embodiments more than 10, and in particular more than 15.

Although polyethylene is the most utilised material, polyolefin mixtures can also be used. In particular, also suitable are polypropylene, polybutene, polystyrene, ethylene/propylene copolymers, ethylene/hexylene copolymers, ethylene/butene copolymers, propylene/butene copolymers, ethylene/propylene/butene copolymers and copolymers of ethylene or propylene with an ethylenically unsaturated monocarboxylic acid, that is to say acrylic acid, methacrylic acid or mixtures thereof.

Suitable fillers and plasticizers are known to the art. In this context, reference is again made to U.S. Pat. No. 3,351,495. A particular filler is finely-divided silica (silicic acid). The average particle size (diameter) of the filler is the range from 0.01 to about 20 .mu.m, the surface area of the filler being in the range from 30 to 950 m²/g, and particularly at least 100 m²/g.

The material to be used according to the invention may comprise a plasticizer, particularly a water-insoluble oil, in particular process oil.

Particularly desirable ranges of amounts for the homogeneous mixture are 15 to 60, preferably 30 to 45, vol. % polyolefin, and 35 to 80, particularly 50 to 65, vol. % filler, and 1 to 10 vol. % plasticizer.

In addition to the constituents mentioned, the material to be used according to the invention can comprise art-recognised additives, such as antioxidants (usually 0.1 to 1%), lubricants (usually 0.1 to 1%), antistatics, pigments, dyestuffs, conductive carbon black, stabilizers, light stabilizers and the like.

A particular membrane is a high density polyolefin/finely-divided silica membrane.

In a further embodiment of the invention, the apparatus according to the invention comprises an end-of-life indicator. Such an embodiment is very useful, as it can inform when the device needs replacing, or when a charge of liquid on the membrane is exhausted. In this invention, this is achieved by a change in colour of the membrane, that is, a membrane charged with liquid is a different colour from a dry membrane. This can be achieved by any convenient means. For example, it may be achieved by the incorporation in the membrane of a material that interacts with the liquid to produce a colour change. The interacting material must naturally be chosen such that the colour change is reversible.

In one embodiment of the invention, the ability to change colour depending on the presence or absence of liquid is an inherent property of the membrane, so that it need not be modified to achieve this. This is a property of the preferred polyolefin/silica battery separator-type membranes hereinabove described, and another reason for their particular utility in the working of this invention. Thus, the invention further provides an apparatus as hereinabove described, in which the need for replenishment of the membrane is indicated by a change in colour of the membrane.

In operation, the liquid is brought into contact with the membrane for a time sufficient to charge it with liquid, and the two are then separated. This can be done, for example, by simply inverting the apparatus and then turning it back again. For example, the membrane may be located near a flat top of the apparatus, such that the apparatus can stand upside-down. Alternatively, the apparatus may be mounted in a cradle or on pivots.

The invention is further described with reference to the accompanying drawing, which depicts a preferred embodiment.

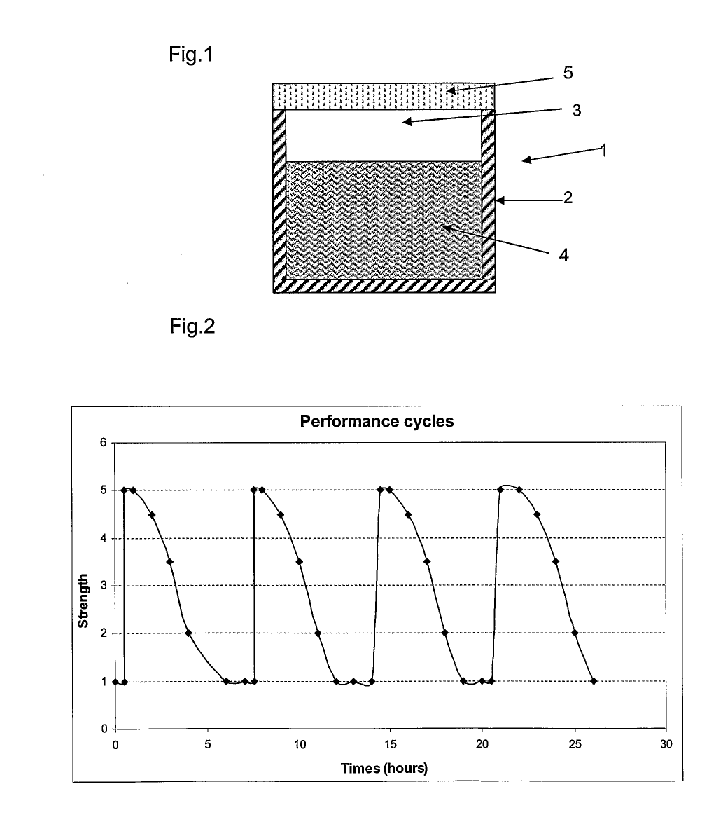

FIG. 1 is a schematic vertical cross-section through a preferred embodiment.

FIG. 2 is a chart, showing the liquid release characteristics of the embodiment over a period of time.

The apparatus, generally indicated as 1 consists of a reservoir 2 that has the shape of a vessel with an opening 3 at one end. The reservoir contains a volatile liquid 4, in this case, a fragrance. The open end of the reservoir is closed with a membrane 5. The membrane is a polyethylene/silica membrane of 1 mm thickness (the actual membrane used is Membrane DS2 drying sweat system ex Daramic, Inc. The membrane 5 has a flat top, which allows the apparatus to be inverted and to sit stably on a horizontal surface.

In operation, the apparatus is inverted and kept in that position for 15 seconds. This allows the membrane to charge with liquid. This is evidenced by a change in colour of the membrane from opaque white to the colour of the liquid phase—the membrane also becomes slightly translucent. The apparatus is set the right way up and volatile liquid commences to emanate from the membrane. The end of life of the particular charge may be observed by observing the colour of the membrane. When it returns to its original colour, it can again be inverted to recharge.

FIG. 2 shows graphically the results of measurements taken over a time period of just over a day, using a fragrance. The strength figures depicted on the ordinate are assessments of the strength of the fragrance taken at intervals by an experienced panel, as used in the fragrance industry. The strengths are 5=very strong, 4=strong, 3=average, 2=weak, 1=very weak, 0=odourless

The membrane is charged, as hereinabove described and allowed to discharge over a period of 7 hours, prior to recharging and reinverting. It can be seen in this particular embodiment that the emission of liquid falls to a low level over the seven hours, but the 15 seconds' charging returns the apparatus to the same level of performance as did the initial charging. This result is repeated in the two subsequent chargings depicted on the graph.

The result is that the user does not become habituated to the fragrance, but perceives it as fresh and new. In addition, the user does not need to continue to utilise the fragrance in such a manner, but can leave the membrane fully discharged for as long as desired, and then return the apparatus to full operating condition by simply inverting to charge the membrane.

The skilled person will perceive many possible variations of this invention, which lie within the scope of the invention.

1. An apparatus adapted to disseminate a volatile liquid into an ambient atmosphere for a desired period of time, the apparatus comprising a reservoir having a single opening to the atmosphere, this opening being closed by a permeable membrane not in contact with the liquid in the reservoir when the apparatus is in operation, the membrane having a thickness of from 0.1-5 mm and being capable of absorbing a quantity of liquid that will evaporate over the desired period when the membrane is brought into direct contact with the liquid.

2. An apparatus according to claim 1, in which the membrane thickness is from 0.5-5 mm.

3. An apparatus according to claim 1, in which the material of the membrane is selected from the group consisting of: polyethylene, polypropylene, polybutene, polystyrene, ethylene/propylene copolymers, ethylene/hexylene copolymers, ethylene/butene copolymers, propylene/butene copolymers, ethylene/propylene/butene copolymers and copolymers of ethylene or propylene with an ethylenically unsaturated monocarboxylic acid; which material is optionally filled.

4. An apparatus according to claim 3, in which the membrane is microporous filled polyolefin.

5. An apparatus according to claim 4, in which the material of the membrane consists essentially of a homogeneous mixture of 8 to 100 vol. % polyolefin having a molecular weight (weight-average) of at least 300,000, a standard load melt index of less than 0.1 and a reduced viscosity of not less than 4.0, 1 to 92 vol. % filler and 1 to 40 vol. % plasticizer.

6. An apparatus according to claim 5, in which the polyolefin is an ultra-high molecular weight polyolefin.

7. An apparatus according to claim 5, in which the polyolefin has a molecular weight of at least 1,000,000.

8. An apparatus according to claim 5, in which the standard load melt index is less than 0.01.

9. An apparatus according to claim 5, in which the reduced viscosity of the polyolefin is more than 10.

10. An apparatus according to claim 3, in which the filler is finely divided silica (silicic acid) having an average particle size (diameter) in the range from 0.01 to about 20 μm, the surface area of the filler being in the range from 30 to 950 m²/g.

11. An apparatus according to claim 4, in which the membrane comprises from 15 to 60 vol. % polyolefin, and 35 to 80 vol. % filler, and 1 to 10 vol. % plasticizer.

12. An apparatus according to claim 1, in which the membrane comprises an end-of-life indicator by means of colour change.

13. An apparatus according to claim 12, in which the material is a polyolefin/silica mixture and the end-of-life colour indication is an inherent property of the material.

14. A method of disseminating a volatile liquid into an ambient atmosphere for a desired time, comprising the application of the liquid to one side of a porous membrane permanently not exposed to the atmosphere, the membrane having a thickness of from 0.1-5 mm and being capable of absorbing sufficient liquid for evaporation from a side of the membrane exposed to the atmosphere over the desired time period.

15. An apparatus according to claim 2, in which the membrane thickness is from 0.2-3 mm.

16. An apparatus according to claim 15, in which the membrane thickness is from 0.6-2 mm.

17. An apparatus according to claim 7, in which the polyolefin has a molecular weight of from 4-7×10⁶.

18. An apparatus according to claim 11, in which the membrane comprises from 30 to 45, vol. % polyolefin, and 35 to 80 vol. % filler, and 1 to 10 vol. % plasticizer.

19. An apparatus according to claim 11, in which the membrane comprises from 15 to 60 vol. % polyolefin, and 50 to 65 vol. % filler, and 1 to 10 vol. % plasticizer.
